(19) United States
(12) Patent Application Publication
(10) Pub. No.: US 2022/0333150 A1
(43) Pub. Date: Oct. 20, 2022

(54) BIOSENSOR, CHANNEL MEMBER USED IN BIOSENSOR, AND METHOD OF USING BIOSENSOR

(71) Applicant: ADVANTEST Corporation, Tokyo (JP)

(72) Inventors: Kiyoto Nakamura, Tokyo (JP); Hirokazu Sanpei, Tokyo (JP)

(73) Assignee: ADVANTEST Corporation, Tokyo (JP)

(21) Appl. No.: 17/764,665

(22) PCT Filed: Sep. 1, 2020
(86) PCT No.: PCT/JP2020/033055

§ 371 Date: Mar. 29, 2022

(30) Foreign Application Priority Data
Nov. 19, 2019 (JP) 2019-208574

(51) Int. Cl. C12Q 1/00; C12Q 1/26; B01L 3/00

(52) U.S. Cl. CPC C12Q 1/006; C12Q 1/26; B01L 3/502715; B01L 2300/0645; B01L 2300/0636; B01L 2300/1827

(57) ABSTRACT

A biosensor includes: a flow channel through which a liquid sample flows, the liquid sample containing a specific component; a holding sheet that is disposed in the flow channel and holds a substance corresponding to the specific component; and a first temperature sensor that is disposed to correspond to the holding sheet and detects a reaction heat generated by a contact reaction between the specific component and the corresponding substance. The biosensor acquires information on the specific component based on the reaction heat.

### CROSS-REFERENCE TO RELATED APPLICATIONS

The present application claims priority from Japanese Patent Application No. 2019-208574 filed on Nov. 19, 2019, the contents of which are incorporated herein by reference in their entirety.

### BACKGROUND

### Technical Field

The present disclosure relates to a biosensor of a calorimetric type, a channel member used in the biosensor, and a method of using the biosensor.

### Description of Related Art

In a biosensor manufactured by a semiconductor manufacturing process, a sensor chip including a temperature sensor and a microchannel are integrally formed, and an enzyme is directly fixed to a hot junction electrode of the temperature sensor provided on the thin film having the cross-linked structure by electrodeposition (refer to, for example, Patent Document 1).

### PATENT DOCUMENT

Patent Document 1: JP2016-173273A

The formation of microchannels by the semiconductor manufacturing process described above is costly and particularly hinders the provision of disposable (so-called disposable type) biosensors. Therefore, in order to provide a disposable biosensor, it is effective to separate a microchannel from a sensor chip and manufacture the microchannel by a method other than a semiconductor manufacturing process.

However, in the case of separating the microchannel from the sensor chip, since the channel wall defining the microchannel is interposed between the enzyme and the temperature sensor, it is difficult for the temperature sensor to detect the reaction heat of the contact reaction between the substrate in the liquid sample and the enzyme.

### SUMMARY

The disclosure provides a biosensor capable of accuracy detecting the reaction heat, a channel member used in the biosensor, and a method of using the biosensor.

[1] A biosensor according to one or more embodiments is a biosensor that acquires information on a specific component on the basis of a reaction heat generated by a contact reaction between the specific component contained in a liquid sample and a corresponding substance corresponding to the specific component, the biosensor including: a flow channel that the liquid sample is able to flow in; a holding sheet that is disposed in the flow channel and holds the corresponding substance; and a first temperature sensor that is disposed to correspond to the holding sheet and detects the reaction heat, in where the corresponding substance is held in the holding sheet.

[2] In one or more embodiments, the holding sheet may include a plurality of communication paths that pass from one surface of the holding sheet to the other surface of the holding sheet.

[3] In one or more embodiments, the holding sheet may be a cloth, paper, a porous body, or a net like body.

[4] In one or more embodiments, the biosensor may include a resin material that contains the corresponding substance, and the resin material may be cured in a state in which the resin material enters the holding sheet.

[5] In one or more embodiments, the biosensor may include: a channel member (i.e., a channel sheet) that the flow channel is formed in; and a sensor member (i.e., a sensor plate) that includes the first temperature sensor, and the channel member and the sensor member may be separatable from each other.

[6] In one or more embodiments, the channel member may include a channel wall that holds the holding sheet and defines the flow channel, the sensor member may include: a sensor chip that includes the first temperature sensor; and a mount member (i.e., a holding film) that the sensor chip is mounted on, the channel wall and the mount member may be interposed between the holding sheet and the first temperature sensor, and the channel wall and the mount member may be not bonded to each other so that the channel member and the sensor member are separatable from each other.

[7] In one or more embodiments, the biosensor may include a fixing device that fixes the channel member and the sensor member, and the fixing device may include a pair of plate-shaped heating members (i.e., heating plates) that sandwich the channel member and the sensor member that are stacked on each other.

[8] In one or more embodiments, the flow channel may include an enlarged part whose inner diameter is enlarged toward the first temperature sensor as compared with other part of the flow channel, and the holding sheet may be disposed in the enlarged part.

[9] In one or more embodiments, the biosensor may include a channel wall that holds the holding sheet and defines the flow channel, a part of an inner surface of the channel wall may be a hydrophilized surface that is subjected to a hydrophilic treatment, and a part of an inner surface of the channel wall in the enlarged part may have hydrophobicity as compared with the hydrophilized surface.

[10] In one or more embodiments, the biosensor may include a channel member that the flow channel is formed in, and the channel member may include: a first film that has a hydrophilized surface; a second film that has a hydrophilized surface and has a first opening in a part of a region corresponding to the flow channel; a spacer that has a groove having a shape corresponding to the flow channel and is attached to the first and second films so as to be interposed between the first and second films; and a third film that has an adhesive layer on one main surface of the third film and is attached to the second film so as to close the first opening, and the holding sheet may be attached to the adhesive layer of the third film.

[11] In one or more embodiments, the biosensor may include a sensor chip that has a beam part that the first temperature sensor is formed in, the sensor chip may include: a board that includes a semiconductor layer forming a part of the first temperature sensor; and an electrode that is disposed on one side of the board and is electrically connected to the first temperature sensor, and a part of the board corresponding to the electrode may be removed so that the electrode is exposed on other side of the board.

[12] In one or more embodiments, the biosensor may include a sensor member that includes the first temperature sensor, and the sensor member may include: a sensor chip that includes the first temperature sensor; a holding film that holds the sensor chip; a wiring board that has a second opening that accommodates the sensor chip, the holding film being attached to the wiring board so as to close the second opening; and a connecting body that connects an electrode of the sensor chip and the wiring of the wiring board.

[13] In one or more embodiments, the biosensor may include a tubular body that is disposed at an inlet of the flow channel so that the tubular body communicates with the flow channel, and an inner hole of the tubular body may have a volume equal to or larger than a volume of the flow channel.

[14] In one or more embodiments, the specific component may be one of a substrate or an enzyme, and the corresponding substance may be other of the enzyme or the substrate.

[15] In one or more embodiments, the biosensor may include a second temperature sensor that is disposed to correspond to a part of the flow channel that the corresponding substance is not disposed in, and the first temperature sensor and the second temperature sensor may be disposed side by side along a width direction of the flow channel.

[16] In one or more embodiments, the biosensor may include: a second temperature sensor that is disposed to correspond to a part of the flow channel that the corresponding substance is not disposed in; and the calculating device that calculates an amount of the specific component on the basis of a first detection result by the first temperature sensor and a second detection result by the second temperature sensor.

[17] A channel member according to one or more embodiments is a channel member that is used in a biosensor that acquires information on a specific component on the basis of a reaction heat generated by a contact reaction between the specific component contained in a liquid sample and a corresponding substance corresponding to the specific component, the channel member including: a flow channel that the liquid sample is able to flow in; and a holding sheet that is disposed in the flow channel and holds the corresponding substance, in which the corresponding substance is held in the holding sheet.

[18] A method of using a biosensor according to one or more embodiments is a method of using the above-described biosensor, including: a first step of closing an outlet of the flow channel; a second step of injecting the liquid sample into the inner hole of the tubular body, a third step of opening the outlet; and a fourth step of detecting the reaction heat by the first temperature sensor.

[19] In one or more embodiments, the third step may be executed after an elapse of a predetermined time from the completion of the second step.

[20] In one or more embodiments, the method may include a fifth step of removing the channel member from the biosensor.

According to the disclosure, since the reaction heat generated by the contact reaction between the specific component and the corresponding substance is stored in the holding sheet and then diffused slowly, it is possible to secure a state long in which the reaction heat can be detected by the temperature sensor, and it is possible to detect the reaction heat with high accuracy.

### BRIEF DESCRIPTION OF THE DRAWINGS

FIG. 1 is a plan view of the overall configuration of the biosensor in one or more embodiments;

FIG. 2 is a front view of the biosensor in one or more embodiments;

FIG. 3 is a cross-sectional view of the biosensor in one or more embodiments and a cross-sectional view taken along III-III line of FIG. 1;

FIG. 4 is an exploded sectional view of the biosensor in one or more embodiments;

FIG. 5 is a plan view of the channel member in one or more embodiments;

FIG. 6 is a cross-sectional view of the channel member in one or more embodiments and a cross-sectional view taken along VI-VI line of FIG. 5;

FIG. 7 is a cross-sectional view of the enlarged part of the channel member in one or more embodiments and is a cross-sectional view taken along VII-VII line of FIG. 6.

FIG. 8 is a cross-sectional view of the enlarged part of the channel member and the sensor chip in one or more embodiments and a cross-sectional view taken along VIII-VIII line of FIG. 7;

FIG. 9 is a cross-sectional view of the modified example of the arrangement of the holding sheets in the enlarged part of the channel member in one or more embodiments;

FIG. 10 is a bottom view of the sensor member in one or more embodiments;

FIG. 11 is a plan view of the sensor chip in one or more embodiments;

FIG. 12 is a cross-sectional view of the sensor chip in one or more embodiments and is a cross-sectional view taken along XII-XII line of FIG. 11;

FIG. 13 is a cross-sectional view taken along A-A line of FIG. 10 and is a diagram showing a state before the sensor chip is attached to the holding film;

FIG. 14 is a cross-sectional view taken along A-A line of FIG. 10 and is a diagram showing a state after forming the connection wiring;

FIG. 15 is a process diagram showing a method of using the biosensor in one or more embodiments;

FIG. 16A is a cross-sectional view of the step S20 of FIG. 15;

FIG. 16B is a cross-sectional view of the step S30 of FIG. 15; and

FIG. 16C is a cross-sectional view of step S40 of FIG. 15.

### DETAILED DESCRIPTION

Hereinafter, embodiments will be described with reference to the drawings.

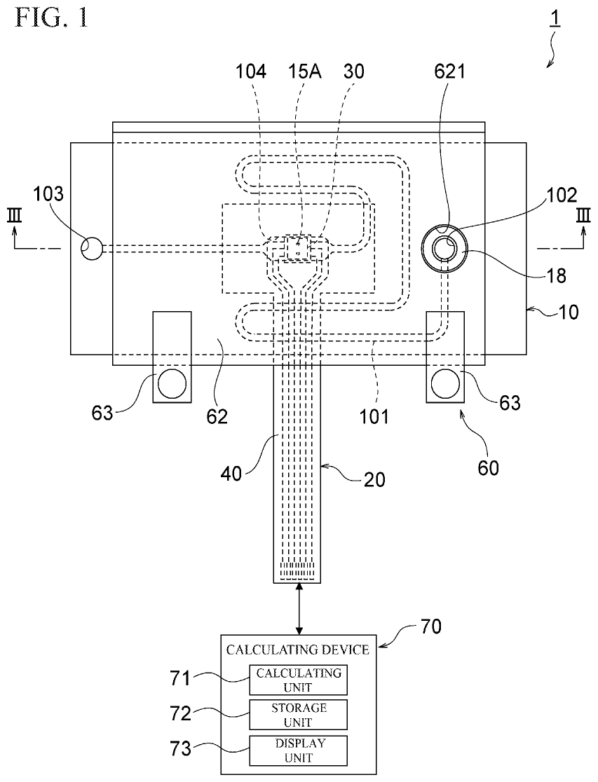

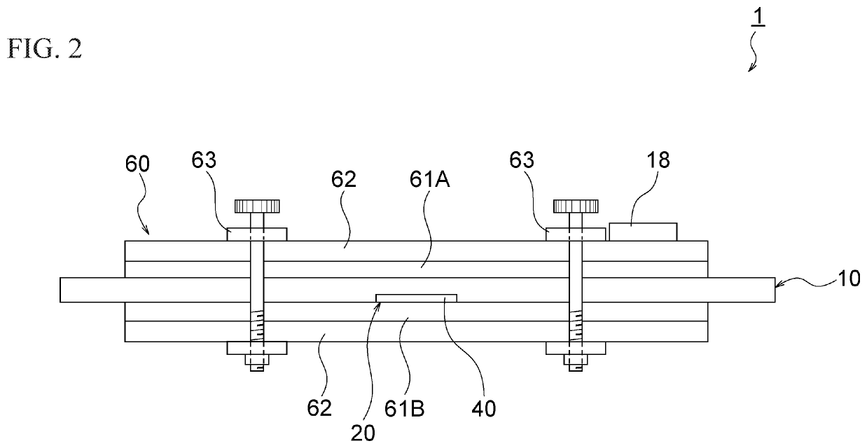

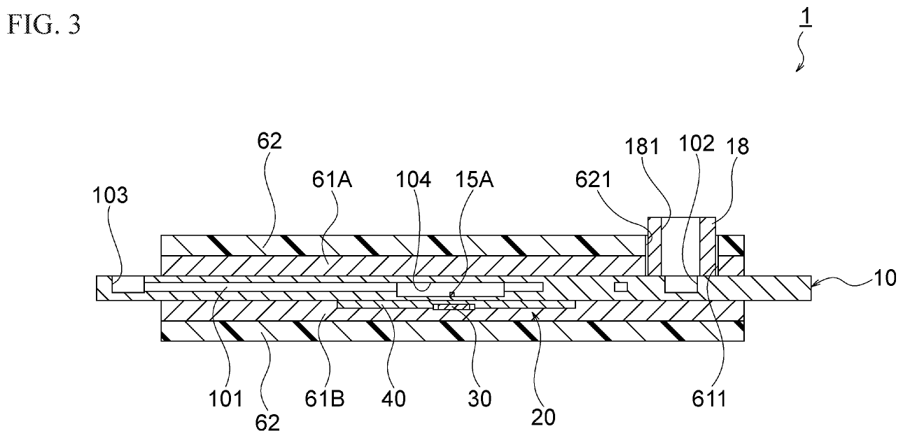

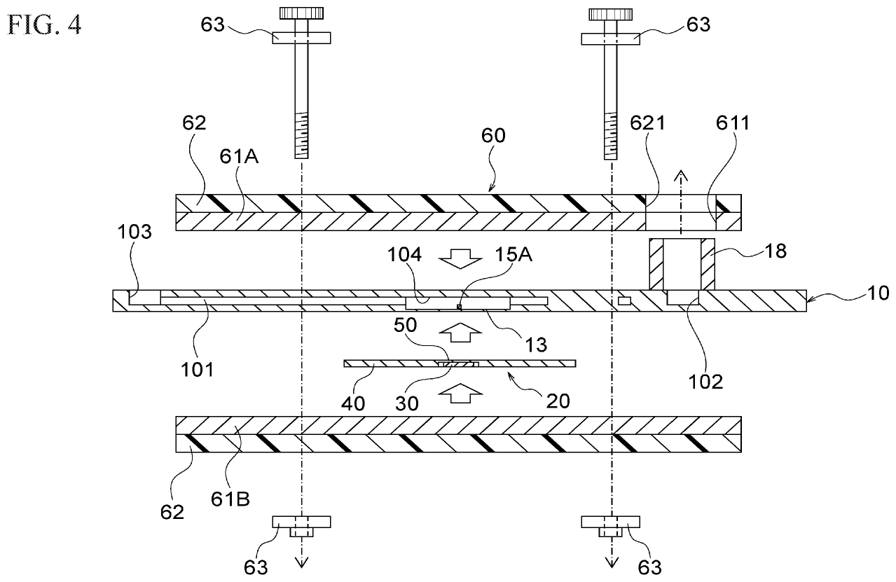

FIG. 1 is a plan view of the overall configuration of the biosensor in one or more embodiments, FIG. 2 is a front view of the biosensor in one or more embodiments, FIG. 3 is a cross-sectional view of the biosensor in one or more embodiments and a cross-sectional view taken along III-III line of FIG. 1, and FIG. 4 is an exploded sectional view of the biosensor in one or more embodiments. In FIG. 4, a clamp 63 is also shown for easy understanding.

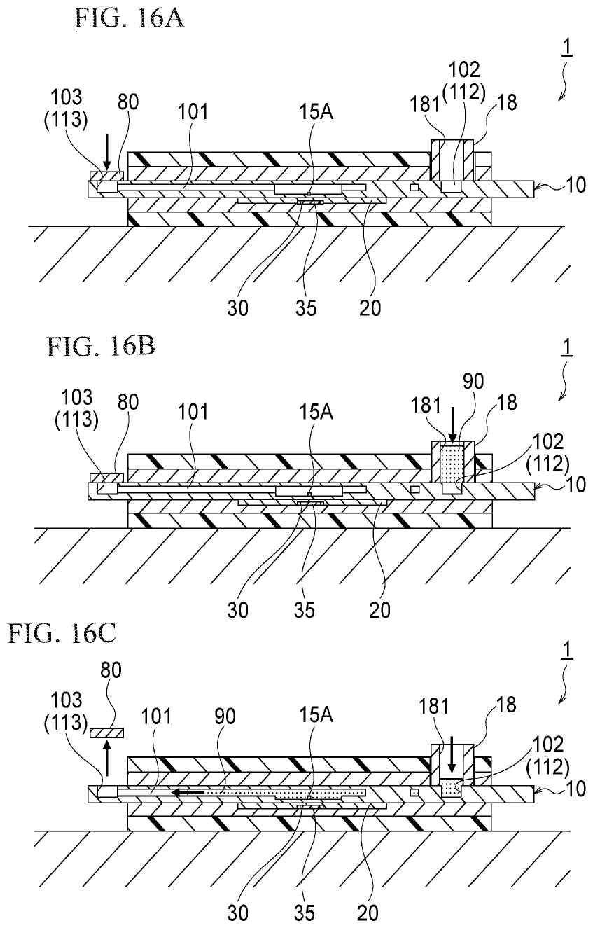

The biosensor 1 in one or more embodiments is a biosensor of a calorimetric type and is a sensor that measures an amount of a substrate contained in a liquid sample 90 (refer to FIG. 16B and FIG. 16C) on the basis of a reaction heat generated in a contact reaction (catalytic reaction) between the substrate and an enzyme. As shown in FIG. 1 to FIG. 4, the biosensor 1 includes a channel member 10, a sensor member 20, a fixing device 60, and a calculating device 70.

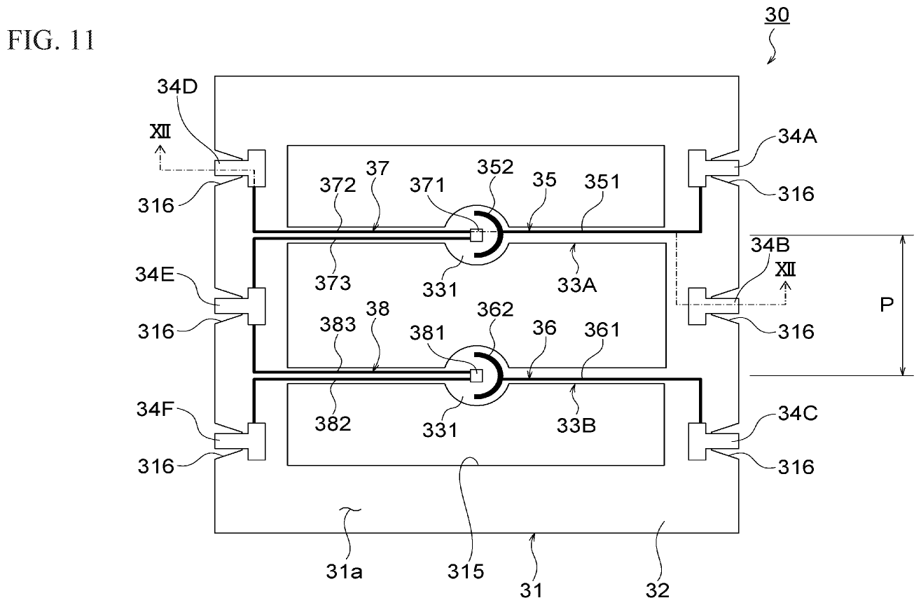

The channel member 10 has a flow channel 101 into which a liquid sample 90 can flow, and the enzyme is fixed in a holding sheet 15A disposed in the flow channel 101. The sensor member 20 may be a plate-like or sheet-like member and has a sensor chip 30 in which a first temperature sensor 35 (refer to FIG. 11) is formed to correspond to the holding sheet 15A. The first temperature sensor 35 detects the temperature of the reaction heat generated by the contact reaction between the enzyme fixed in the holding sheet 15A and the substrate contained in the liquid sample 90. The channel member 10 and the sensor member 20 stacked on each other are detachably fixed by the fixing device 60. The calculating device 80 calculates the amount of the substrate on the basis of the output signal from the first temperature sensor 35.

As a specific example of the liquid sample 90, a body fluid obtained from a human such as blood, urine, sweat, saliva, and tear can be exemplified. As long as the liquid sample 90 is a liquid derived from a living body, it is not particularly limited to the above and may be, for example, a body fluid obtained from an animal such as a dog and a cat.

Specific examples of the substrate are not particularly limited, and examples thereof include glucose, uric acid, lactic acid, protein, fat, creatinine, and bilirubin. Specific examples of the enzyme include glucose oxidase, peroxidase, lactate oxidase, trypsin, lipase, creatininase, and bilirubin oxidase.

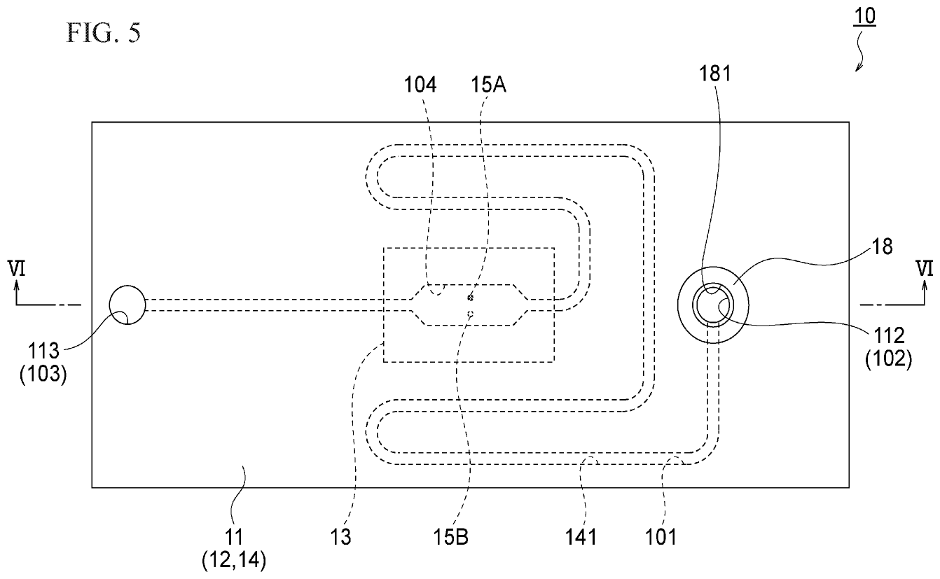

Hereinafter, a configuration of each member constituting the biosensor 1 will be described. First, the configuration of the channel member 10 will be described with reference to FIG. 5 to FIG. 9.

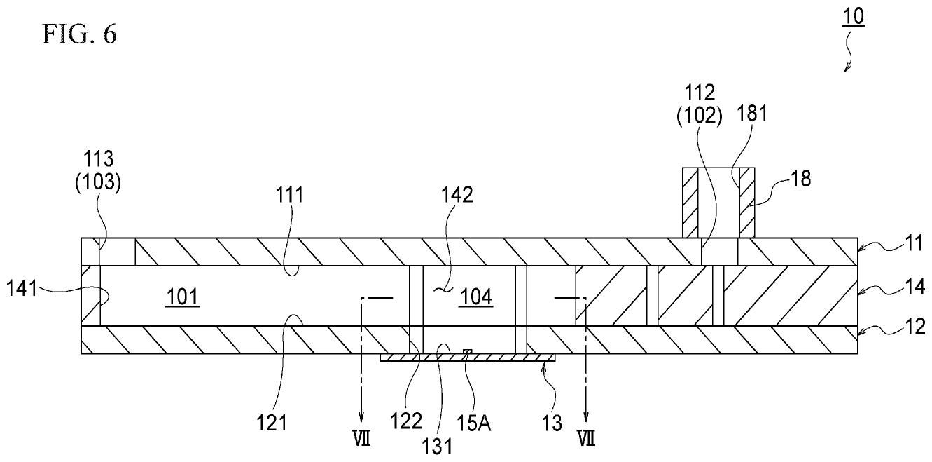

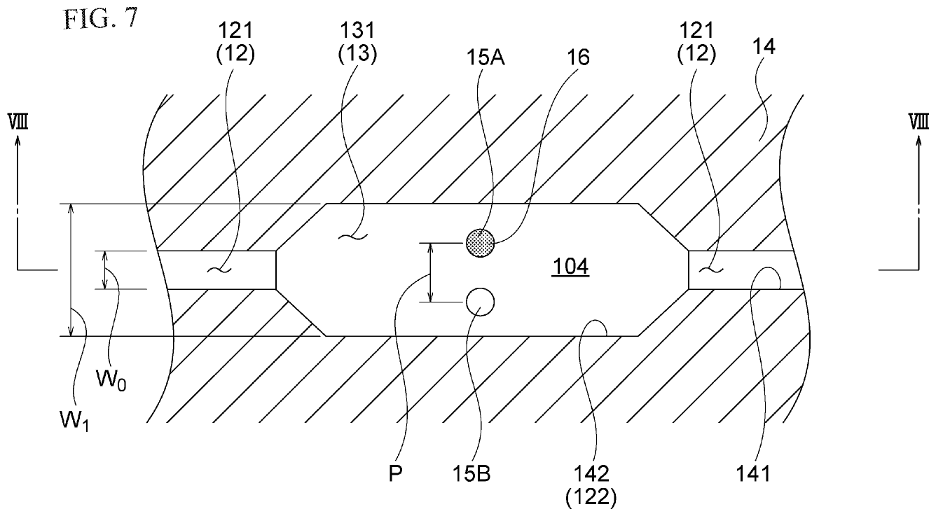

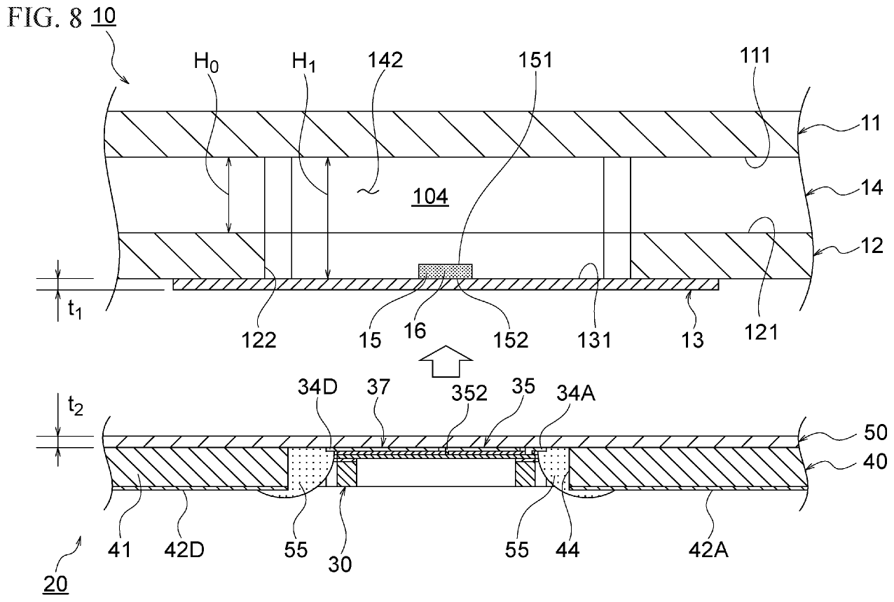

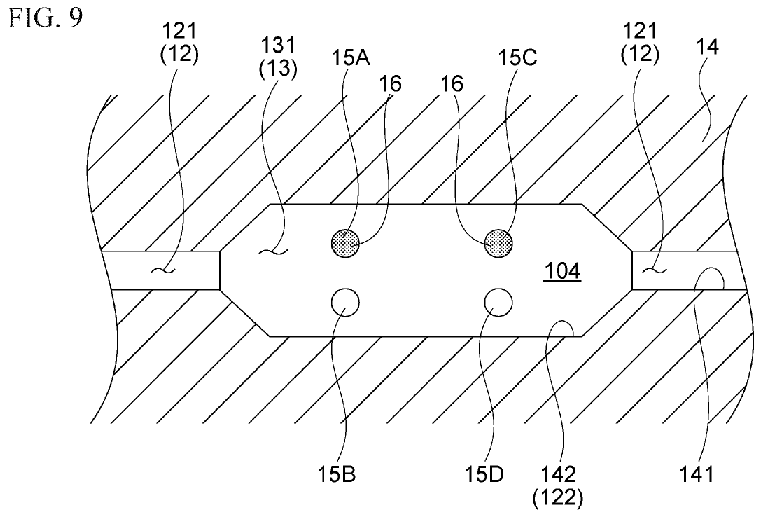

FIG. 5 is a plan view of the channel member in one or more embodiments, FIG. 6 is a cross-sectional view of the channel member in one or more embodiments and a cross-sectional view taken along VI-VI line of FIG. 5, FIG. 7 is a cross-sectional view of the enlarged part of the channel member in one or more embodiments and is a cross-sectional view taken along VII-VII line of FIG. 6, FIG. 8 is a cross-sectional view of the enlarged part of the channel member and the sensor chip in one or more embodiments and a cross-sectional view taken along VIII-VIII line of FIG. 7, and FIG. 9 is a cross-sectional view of the modified example of the arrangement of the holding sheets in the enlarged part of the channel member in one or more embodiments.

As shown in FIG. 5 and FIG. 6, the channel member 10 is a sheet-like member that a flow channel 101 that the liquid sample 90 can flow in is formed in. The holding sheet 15A that holds the enzyme and a holding sheet 15B that does not hold an enzyme are disposed in the flow channel 101. That is, the enzyme is fixed in the holding sheet 15A, whereas an enzyme is not fixed in the holding sheet 15B.

In one or more embodiments, the flow channel 101 has a planar shape extending linearly with a substantially constant width. An inlet 102 is disposed at one end (the right end in the figure) of the flow channel 101, an outlet 103 is disposed at the other end (the left end in the figure) of the flow channel 101, and an enlarged part 104 is disposed between the inlet 102 and the outlet 103.

As shown in FIG. 7, the enlarged part 104 has a width W₁ wider than the width W₀ of the other part of the flow channel 101 (W₁>W₀). Further, as shown in FIG. 8, the enlarged part 104 has a height H₁ higher than the height H₀ of the other part of the flow channel 101 (H₁>H₀). The holding sheets 15A and 15B are disposed in the enlarged part 104.

In the one or more embodiments, since the flow channel between the inlet 102 and the enlarged part 104 meanders, the distance from the inlet 102 to the enlarged part 104 is longer than the distance from the enlarged part 104 to the outlet 103. As a result, it is possible to adjust the temperature of the liquid sample 90 to the temperature of the channel member 10 until the liquid sample 90 that has entered the flow channel 101 from the inlet 102 reaches the enlarged part 104. The planar shape of the flow channel 101 is not particularly limited to the shape shown in FIG. 5 and may be any shape.

As shown in FIG. 5 and FIG. 6, the channel member 10 includes first to third films 11 to 13, a spacer 14, and holding sheets 15A and 15B.

The first film 11 is a film made of a resin material such as polyester and has a thickness of, not particular limited, about 100 μm. The lower surface (the surface facing the spacer 14) of the first film 11 is a hydrophilized surface 111 that is subjected to a hydrophilic treatment on the entire surface thereof. As a specific example of the hydrophilic treatment, coating of a surfactant or a hydrophilic polymer on the lower surface of the first film 11, a plasma treatment or the like can be exemplified.

Similarly to the first film 11 described above, the second film 12 is also a film made of a resin material such as polyester and has a thickness of, not particular limited, about 100 μm. The upper surface (the surface facing the spacer 14) of the second film 12 is also a hydrophilized surface 121 that is subjected to hydrophilic treatment on the entire surface thereof.

The spacer 14 is interposed between the first film 11 and the second film 12. The spacer 14 is a film made of a resin material such as polyethylene terephthalate (PET) and has a thickness of, not particular limited, about 500 μm. A groove 141 having a planar shape corresponding to the above-mentioned flow channel 101 is formed in the spacer 14. As shown in FIG. 7, the width W₁ of the wide part 142 of the groove 141 corresponding to the enlarged part 104 is wider than the width W₀ of the other part of the groove 141 (W₁>W₀).

As shown in FIG. 6 and FIG. 8, the first film 11 is attached to the upper surface of the spacer 14 via an adhesive layer (not shown). A part of the hydrophilized surface 111 of the first film 11 facing the groove 141 is exposed to the flow channel 101. A pair of openings 112 and 113 are formed in the first film 11 to face both ends of the groove 141, and the respective openings 112 and 113 function as the inlet 102 and the outlet 103 of the above-mentioned flow channel 101.

Similarly, the second film 12 is also attached to the lower surface of the spacer 14 via an adhesive layer (not shown). A part of the hydrophilized surface 121 of the second film 12 facing the groove 141 is exposed to the flow channel 101. An opening 122 is formed in the second film 12 at a part facing the wide part 142 of the groove 141 of the spacer 14. Therefore, the inner diameter of the flow channel 101 is expanded downward (on the sensor member 20 side) at the enlarged part 104 as compared with the other part of the flow channel 101. As shown in FIG. 7, the opening 122 has the same width W₁ as the wide part 142 of the groove 141.

The third film 13 is a film made of a resin material such as polyester. The thickness t₁ of the third film 13 is, not particularly limited, about 16 μm (t₁=16 μm). As shown in FIG. 6 and FIG. 8, the adhesive layer 131 is formed on the entire upper surface (the surface facing the second film 12) of the third film 13. The third film 13 is attached to the lower surface of the second film 12 to close the opening 122 of the second film 12.

The two holding sheets 15A and 15B are disposed on the third film 13. The enzyme is fixed in the holding sheet 15A on one side (upper side in FIG. 7), whereas the enzyme is not fixed in the holding sheet 15B on the other side (lower side in FIG. 7). The holding sheets 15A and 15B are disposed side by side at intervals along the width direction of the flow channel 101. More specifically, when cutting along the width direction of the flow channel 101, the holding sheets 15A and 15B are arranged in the same opening of the flow channel 101 at predetermined intervals along the width direction of the flow channel 101. The pitch P (distance between centers) between the holding sheets 15A and 15B is substantially the same as the pitch P between the beam portions 33A and 33B of the sensor chip 30 described later. Hereinafter, the holding sheets 15A and 15B are also collectively referred to as “holding sheet 15”.

The holding sheet 15 is a sheet piece having a plurality of communication paths that pass from one surface 151 of the holding sheet 15 to the other surface 152 of the holding sheet 15. Specific examples of the holding sheet 15 are not particularly limited, but a nonwoven fabric made of cupra fibers (Bencot (registered trademark) PS-2 manufactured by Asahi Kasei Corp.) can be exemplified, and has, for example, a circular shape having a diameter of about 1 mm and a thickness of about 30 μm. Alternatively, although not particularly limited, a non-woven fabric wiper such as a wiper manufactured by Nippon Paper Clasia Co., Ltd. may be used as the holding sheet 15. Alternatively, a non-woven fabric other than the above may be used as the holding sheet 15, or a cloth other than the non-woven fabric may be used.

As the holding sheet 15, paper may be used instead of the non-woven fabric. Specific examples of the paper that can be used as the holding sheet 15 include non-woven paper, filter paper, blotter paper, Japanese paper, and the like. Although not particularly limited, a paper wiper such as KimWipes manufactured by Nippon Paper Clasia Co., Ltd. can be used as the holding sheet 15. By configuring the holding sheet 15 with such cloth or paper, it is possible to reduce the cost of the holding sheet 15.

Alternatively, as the holding sheet 15, a porous body or a net like body may be used instead of the cloth and paper. As a specific example of the porous body that can be used as the holding sheet 15, a sponge having an open cell structure can be exemplified. Further, as a net-like body that can be used as the holding sheet 15, a mesh member formed by knitting a fine metal wire of about 10 μm can be exemplified.

Alternatively, as the holding sheet 15, a sheet piece that can absorb water by a capillary phenomenon other than the above-mentioned cloth, paper, porous body, or net like body may be used.

The enzyme is fixed to one of the holding sheets 15A. Specifically, the resin material 16 (see FIG. 7) containing an enzyme is cured in a state where the resin material 16 (see FIG. 7) containing an enzyme enters the communication paths of the holding sheet 15A. Therefore, the enzyme is held in the holding sheet 15A to be exposed to the outside of the holding sheet 15A and also to be present inside the holding sheet 15A.

The enzyme held in the holding sheet 15A is an enzyme contained in the liquid sample 90 and corresponding to the substrate to be detected. Specifically, when the substrate contained in the liquid sample 90 is glucose, the enzyme corresponding to the glucose is glucose oxidase.

Similarly, the enzyme corresponding to uric acid is peroxidase, the enzyme corresponding to lactic acid is lactic acid oxidase, the enzyme corresponding to protein is trypsin, the enzyme corresponding to fat is lipase, the enzyme corresponding to creatinin is creatinase, and the enzyme corresponding to bilirubin is bilirubin oxidase.

In particular, as a method for measuring albumin that is a main component of proteins contained in urine, (1) a protein error method based on a pH value, (2) a latex agglutination turbidimetric immunoassay method, and (3) an enzyme sorbent method (ELISA method) are known. However, in the methods (1) and (2) described above, an error occurs when a substance similar to albumin is contained in urine, and in the method (3) described above, the measurement work is complicated and expensive. On the other hand, by measuring albumin with the calorimetric method using trypsin as an enzyme, it is possible to perform the measurement of albumin with high accuracy by eliminating the influence of substances similar to albumin contained in urine, and it is possible to perform the measurement of albumin at low cost in a short time.

In one or more embodiments, first, an aqueous solution containing an enzyme and a resin material is impregnated into this holding sheet 15A. Specific examples of such a resin material are not particularly limited, but a photocurable polyvinyl alcohol (BIOSURFINE (registered trademark)-AWP manufactured by Toyo Gosei Co., Ltd.) can be exemplified. Next, after the aqueous solution is dried in a state in which the resin material containing the enzyme enters the inside of the holding sheet 15A through the communication paths of the holding sheet 15A, the resin material is cured by irradiating ultraviolet rays, thereby holding the enzyme in the holding sheet 15A. The enzyme may be directly held in the holding sheet 15A by allowing the enzyme alone to enter the holding sheet 15A without using such a resin material.

As shown in FIG. 9, in addition to the pair of holding sheets 15A and 15B, another pair of holding sheets 15C and 15D may be disposed in the enlarged part 104 of the flow channel 101. An enzyme different from the enzyme fixed in the holding sheet 15A is fixed in one holding sheet 15C. On the other hand, like the holding sheet 15B, the enzyme is not fixed in the other holding sheet 15D. In this case, in addition to the sensor chip 30 (described later) corresponding to the pair of holding sheets 15A and 15B, the sensor member includes another sensor chip corresponding to the pair of holding sheets 15C and 15D.

The number of kinds of enzymes fixed in the flow channel 101 is not limited to the above-mentioned one or two kinds, and three or more kinds of enzymes may be fixed in the flow channel 101. In this case, the number of sets of holding sheets corresponding to the number of kinds of the enzyme is disposed in the flow channel 101.

Returning to FIG. 5 to FIG. 8, the holding sheets 15A and 15B are held by the third film 13 in a state where the holding sheets 15A and 15B are attached to the adhesive layer 131 of the third film 13 described above. That is, in one or more embodiments, the adhesive layer 131 for fixing the third film 13 to the second film 12 is also used for holding the holding sheets 15A and 15B on the third film 13. The holding sheets 15A and 15B are firmly fixed to the third film 13 by the adhesive layer 131.

Here, as described above, the first film 11 has a hydrophilized surface 111 on the entire lower surface thereof, and a part of the hydrophilized surface 111 facing the groove 141 is exposed in the flow channel 101. Similarly, the second film 12 also has a hydrophilized surface 121 on the entire upper surface thereof, and a part of the hydrophilized surface 121 facing the groove 141 is exposed in the flow channel 101. Since the hydrophilized surfaces 111 and 121 are exposed to the flow channel 101 in this way, it is possible to allow the liquid sample 90 to be positively flowed from the inlet 102 to the outlet 103 in the flow channel 101 by utilizing the capillary phenomenon.

On the other hand, as described above, the third film 13 has an adhesive layer 131 on the entire upper surface thereof, and a part of the adhesive layer 131 facing the opening 122 is exposed in the flow channel 101. In general, since the adhesive layer has hydrophobicity as compared with the hydrophilized surface, the adhesive layer 131 also has hydrophobicity as compared with the hydrophilized surfaces 111 and 121. Due to the hydrophobicity of the adhesive layer 131, it is possible to slow down the flow velocity of the liquid sample 90 around the holding sheets 15A and 15B, and it is possible to improve the detection accuracy of the biosensor 1. As described above, in one or more embodiments, the adhesive layer 131 for fixing the third film 13 to the second film 12 is used as a hydrophobic surface in addition to fixing the holding sheets 15A and 15B.

Instead of the adhesive layer 131, the upper surface of the third film 13 may be subjected to a silane coupling treatment or a fluorine plasma treatment to form a hydrophobic treatment surface on the third film 13.

Further, the channel member 10 of one or more embodiments has a tubular body 18 having a cylindrical shape as shown in FIG. 5 and FIG. 6. The tubular body 18 is disposed on the first film 11 to face the inlet 102 of the flow channel 101, and the inner hole 181 of the tubular body 18 communicates with the flow channel 101. The inner hole 181 of the tubular body 18 has a volume equal to or larger than the volume of the flow channel 101. Therefore, the tubular body 18 can hold the entire amount of the liquid sample 90 before flowing into the flow channel 101.

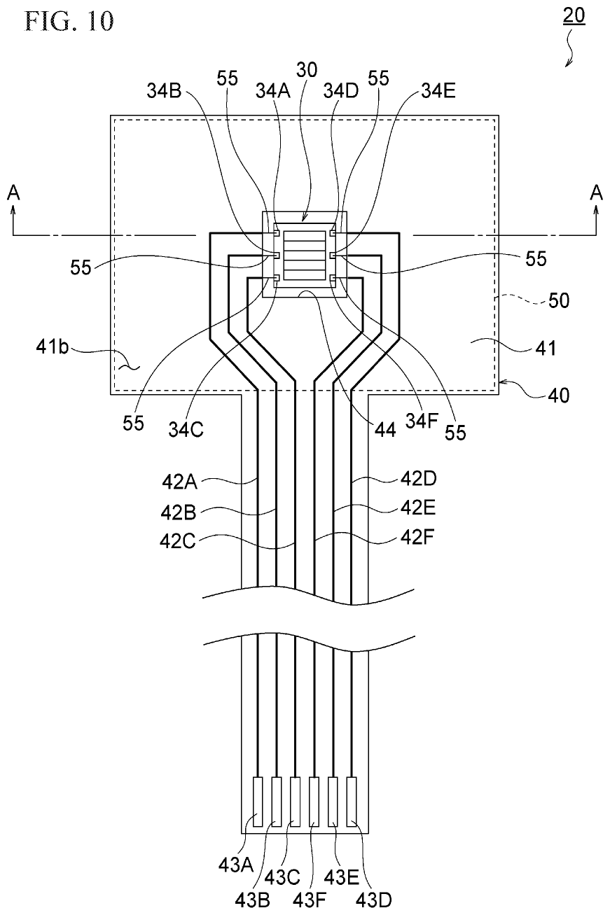

Next, the sensor member 20 will be described with reference to FIG. 10 to FIG. 14.

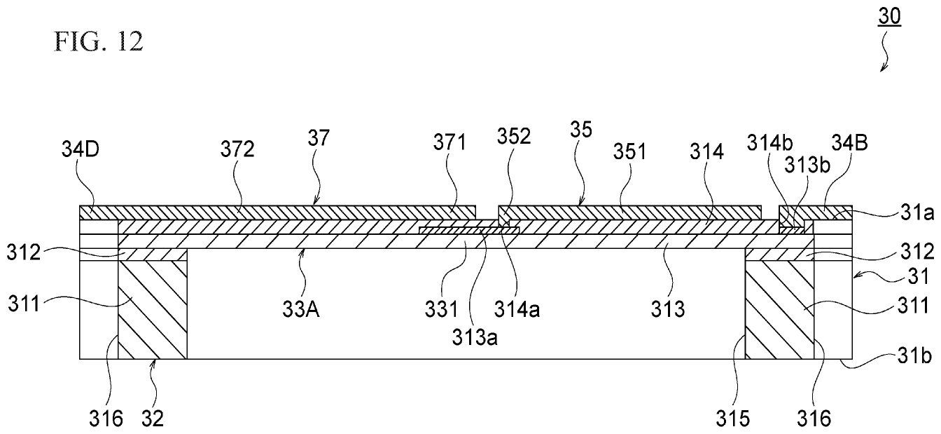

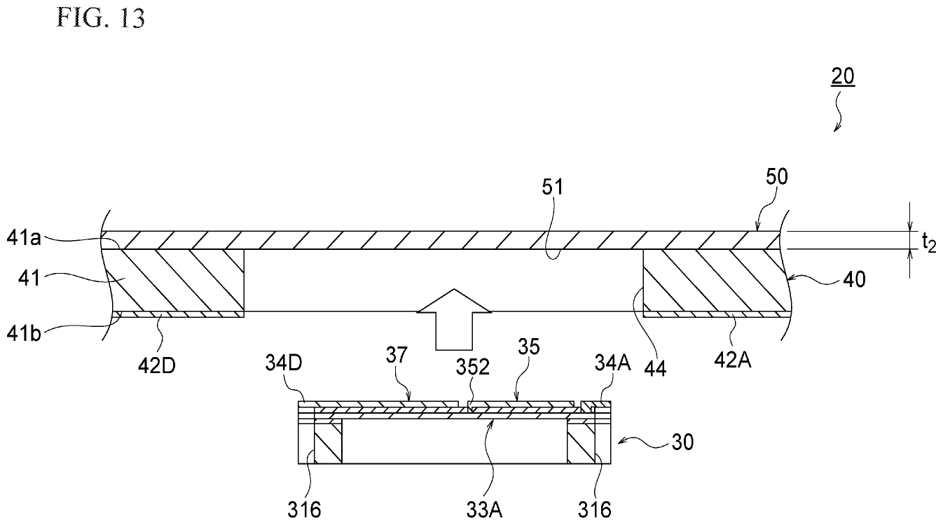

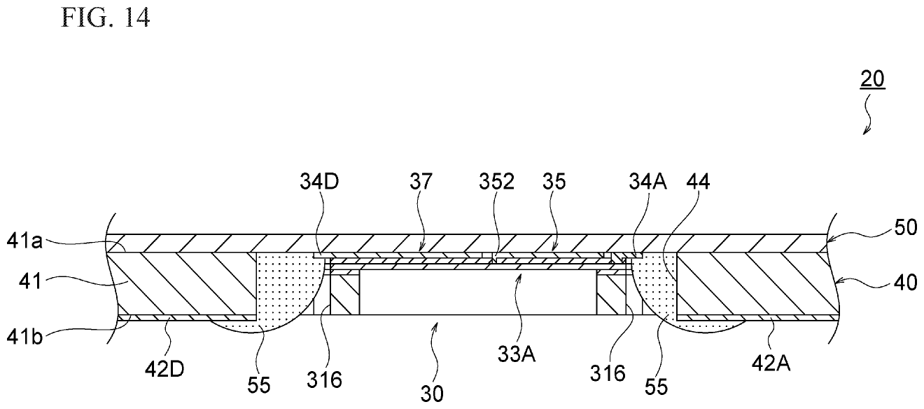

FIG. 10 is a bottom view of the sensor member in one or more embodiments, FIG. 11 is a plan view of the sensor chip in one or more embodiments, FIG. 12 is a cross-sectional view of the sensor chip in one or more embodiments and is a cross-sectional view taken along XII-XII line of FIG. 11, FIG. 13 is a cross-sectional view taken along A-A line of FIG. 10 and is a diagram showing a state before the sensor chip is attached to the holding film, and FIG. 14 is a cross-sectional view taken along A-A line of FIG. 10 and is a diagram showing a state after forming the connection wiring.

As shown in FIG. 10, the sensor member 20 includes a sensor chip 30, a wiring board 40, a holding film 50, and connection wirings 55.

As shown in FIG. 11 and FIG. 12, the sensor chip 30 is a chip including first and second temperature sensors 35 and 36, and first and second heaters 37 and 38. The sensor chip 30 is formed by processing an SOI substrate using a known MEMS technique. The sensor chip 30 may include an absolute temperature sensor that detects the absolute temperature.

The substrate 31 constituting the sensor chip 30 includes a first Si layer 311, a first SiO₂ layer 312, a second Si layer 313, and a second SiO₂ layer 314. Ohmic electrodes 313a and 313b are formed in predetermined regions of the second Si layer 313 (regions corresponding to the enlarged parts 331 of the beam parts 33A and 33B and the through hole 314b of the second SiO₂ layer 314 described later). The ohmic electrodes 313a and 313b are formed by doping the predetermined regions of the second Si layer 313 with a dopant such as phosphorus or boron by ion implantation, forming an aluminum film on the predetermined regions, and alloying the aluminum film with the second Si layer 313 by heating the aluminum film. The insulating layer formed on the second Si layer 313, as long as the insulating layer having excellent processability, is not particularly limited to the second SiO₂ layer 314.

An opening 315 penetrating from the lower surface 31b to the upper surface 31a of the substrate 31 is formed at the center of the substrate 31 by etching or the like, thereby a rectangular frame portion 32 is formed. Further, when the opening 315 is formed in the substrate 31, a part of the second Si layer 313 and a part of the second SiO₂ layer 314 remain without being etched, thereby a pair of beam parts 33A and 33B that are spanned in the frame part 32 are formed. Specific examples of such etching include Deep RIE and wet etching.

The beam parts 33A and 33B extend substantially in parallel with each other. The pitch P between the beam parts 33A and 33B is substantially the same as the pitch P between the holding sheets 15A and 15B described above. The beam parts 33A and 33B have a circular enlarged part 331 at substantially the center of the beam parts 33A and 33B respectively. The shape of the enlarged part 331 is not particularly limited to a circular shape. Alternatively, the beam parts 33A and 33B may not have the enlarged parts 331.

Further, conductive wires 351 and 361, heat generating parts 371 and 381, wirings 372, 373, 382 and 383, and electrodes 34A to 34F are formed on the upper surface 31a of the substrate 31.

The conductive wires 351 and 361, the heat generating parts 371 and 381, the wirings 372, 373, 382 and 383, and the electrodes 34A to 34F are composed of conductive thin films formed on the second SiO₂ layer 314 by sputtering, vapor deposition, plating, or the like. In one or more embodiments, the conductive wire 351 and 361, the heat generating parts 371 and 381, the wiring 372, 373, 382 and 383, and the electrodes 34A to 34F are all composed of a titanium (Ti) layer and a gold (Au) layer formed on the titanium layer. The materials constituting the conductive wires 351 and 361, the heat generating parts 371 and 381, the wirings 372, 373, 382 and 383, and the electrodes 34A to 34F are not particularly limited to the above described as long as they have conductivity.

The first conductive wire 351 extends from the enlarged part 331 of the first beam part 33A to the first electrode 34A. The tip part 352 of the first conductive wire 351 has a substantially U-shape (shape of a semicircular arc) and is connected to the second Si layer 313 through the through hole 314a formed in the second SiO₂ layer 314. At this time, the ohmic electrode 313a is interposed between the tip part 352 of the first conductive wire 351 and the second Si layer 313. Further, the second Si layer 313 is connected to the second electrode 34B through the through hole 314b formed in the second SiO₂ layer 314. At this time, the ohmic electrode 313b is interposed between the second Si layer 313 and the second electrode 34B.

The first temperature sensor 35 includes a thermocouple composed of a first conductive wire 351 as a first heat conductor and a second Si layer 313 as a second heat conductor. The ohmic electrode 313a connecting the tip part 352 of the first conductive wire 351 and the second Si layer 313 functions as a hot junction (measuring junction) of the first temperature sensor 35. The second electrode 34B connected to the second Si layer 313 functions as a cold junction (reference junction) of the first temperature sensor 35.

Similarly, the second conductive wire 361 extends from the enlarged part 331 of the second beam part 33B to the third electrode 34C. The tip part 362 of the second electric wire 361 also has a substantially U-shape (shape of a semicircular arc) and is connected to the second Si layer 313 through a through hole formed in the second SiO₂ layer 314. At this time, an ohmic electrode is interposed between the tip part 362 of the second conductive wire 361 and the second Si layer 313.

The second temperature sensor 36 includes a thermocouple composed of a second conductive wire 361 as a first heat conductor and a second Si layer 313 as a second heat conductor. The ohmic electrode connecting the tip part 362 of the second conductive wire 361 and the second Si layer 313 functions as a hot junction (measuring junction) of the second temperature sensor 36. The second electrode 34B connected to the second Si layer 313 functions as a cold junction (reference junction) of the second temperature sensor 36. That is, the second electrode 34B functions as a common cold junction (common reference junction) for the first and second temperature sensors 35 and 36.

The first heater 37 includes a first heating part 371 and wirings 372 and 373. The first heating part 371 is disposed in the enlarged part 331 of the first beam part 33A to be surrounded by the tip part 352 of the first conductive wire 351. A pair of wirings 372 and 373 are connected to the first heating part 371. One wiring 372 extends from the first heating part 371 to the fourth electrode 34D, while the other wiring 373 extends from the first heating part 371 to the fifth electrode 34E.

Similarly, the second heater 38 includes a second heating part 381 and wirings 382 and 383. Similarly to the first heating part 371 described above, the second heating part 381 is also disposed in the enlarged part 331 of the second beam part 33B to be surrounded by the tip part 362 of the second conductive wire 361. A pair of wirings 382 and 383 are connected to the second heating part 381. One wiring 382 extends from the second heating part 381 to the sixth electrode 34F, while the other wiring 383 extends from the second heating part 381 to the fifth electrode 34E. That is, the fifth electrode 34E functions as a common electrode of the first and second heaters 37 and 38.

The electrodes 34A to 34F are arranged on the outer peripheral part of the frame portion 32. As shown in FIG. 12, a part of the substrate 31 facing the electrode 34A is removed by etching or the like, and a notch 316 is formed in the substrate 31. Similarly, the parts of the substrate 31 facing the electrodes 34B to 34F are removed by etching or the like, and notches 316 are formed in the substrate 31. Each of the electrodes 34A to 34F is exposed from the lower surface 31b of the substrate 31 via the notch 316.

The configuration of the sensor chip 30 is not particularly limited to the above. For example, an insulating film (for example, a SiO₂ layer) may be further formed on the second SiO₂ layer 314 to cover the conductive wires 351 and 361, the heat generating parts 371 and 381, and the wirings 372, 373, 382 and 383, and the electrodes 34A to 34F may be formed on the insulating film. In this case, a plurality of through holes are formed in the insulating film, through these through holes, the electrode 34A and the conductive wire 351 are connected, the electrode 34B and the ohmic electrode 313b are connected, the electrode 34C and the conductive wire 361 are connected, the electrode 34D and the wiring 372 are connected, the electrode 34E and the wiring 373 and 383 are connected, and the electrode 34F and the wiring 382 are connected.

Alternatively, instead of the semiconductor substrate, a sensor chip having a thermocouple may be configured by forming a pair of metal wires on a resin substrate having a beam part. The above-mentioned beam parts 33A and 33B are both end support beams supported by the frame part 32, but are not particularly limited, and the beam parts 33A and 33B may be single end support beams. Instead of the thermocouple, the thermopile may be used as the first and second temperature sensors 35 and 36. Also in this case, the first and second temperature sensors 35 and 36 are arranged so that the measuring junctions of the thermopiles face the holding sheets 15A and 15B.

The wiring board 40 is a so-called flexible printed wiring board (FPC). As shown in FIG. 10, the wiring board 40 includes a base film 41 and wirings 42A to 42F disposed on the base film 41. The wiring board 40 has an opening 44 large enough to accommodate the sensor chip 30. As shown in FIG. 13 and FIG. 14, the wirings 42A to 42F are formed on the lower surface 41b of the base film 41.

The holding film 50 is a film made of a resin material such as polyester. The thickness t₂ of the holding film 50 is not particularly limited, but is about 16 μm (t₂=16 μm). The holding film 50 has an adhesive layer 51 formed on the lower surface of the holding film 50. The holding film 50 is attached to the upper surface 41a of the base film 41 of the wiring board 40 so as to close the opening 44. The sensor chip 30 is housed in the opening 44 of the wiring board 40 in a state in which the sensor chip 30 is held by the adhesive layer 51 of the holding film 50.

As shown in FIG. 10, the wirings 42A to 42F of the wiring board 40 are respectively disposed to correspond to the electrodes 34A to 34F of the sensor chip 30. As shown in FIG. 10 and FIG. 14, one end of the wiring 42A is connected to the electrode 34A via the connection wiring 55.

The connection wiring 55 is formed by printing a conductive paste from the wiring 42A to the electrode 34A and curing the conductive paste. At this time, since the electrode 34A is exposed from the lower surface 31b of the substrate 31 via the notch 316, the connection wiring 55 is connected to the electrode 34A from the lower surface 31b side of the substrate 31. Further, since the electrode 34A is surrounded by the notch 316, it is possible to suppress the spread of the conductive paste, and it is possible to suppress the generation of unnecessary leaks due to the protrusion of the conductive paste.

The corresponding wirings 42B to 42F and the electrodes 34B to 34F are also individually connected via the connection wirings 55. The connection wiring 55 is connected to each of the electrodes 34B to 34F from the lower surface 31b side of the substrate 31. The wirings 42A to 42F extend to the ends of the wiring boards 40, and terminals 43A to 43F are respectively disposed at the other ends of the wirings 42A to 42F.

As another method of exposing the electrodes from the lower surface of the substrate, a method of forming a through hole inside the substrate and a method of forming a lead wiring on the side wall of the substrate can be exemplified. However, as compared with these methods, in the method of one or more embodiments using the notch 316, it is possible to expose the electrodes 34A to 34F to the lower surface 31b of the substrate 31 at low cost, and it is possible to easily connect the wirings 42A to 42F and the electrodes 34A to 34F.

As shown in FIG. 4 and FIG. 8, the channel member 10 and the sensor member 20 described above are overlapped with each other so that the third film 13 and the holding film 50 are in contact with each other. At this time, the channel member 10 and the sensor member 20 are overlapped with each other so that the enlarged parts 331 of the beam portions 33A and 33B of the sensor chip 30 respectively face the holding sheets 15A and 15B disposed in the enlarged part 104 of the flow channel 101.

Therefore, the measuring junction 313a of the first temperature sensor 35 faces the holding sheet 15A, and the first temperature sensor 35 can detect the temperature of the holding sheet 15A. Similarly, the measuring junction of the second temperature sensor 36 faces the holding sheet 15B, and the second temperature sensor 36 can detect the temperature of the holding sheet 15B.

Further, the heat generating part 371 of the first heater 37 faces the holding sheet 15A, and the first heater 37 can heat the holding sheet 15A. Similarly, the heat generating part 381 of the second heater 38 faces the holding sheet 15B, and the second heater 38 can heat the holding sheet 15B. The first and second heaters 37 and 38 are used, for example, for checking the basic characteristics of the biosensor 1.

At this time, as described above, the opening 122 is formed in the second film 12 of the channel member 10, and the inner diameter of the flow channel 101 is enlarged downward (toward sensor member 20) at the enlarged part 104 as compared with the other part of the flow channel 101. In other words, the channel walls 12 and 13 between the flow channel 101 and the sensor member 20 are the thinnest at the enlarged part 104. Therefore, it is possible to accurately detect the minute reaction heat generated by the contact reaction between the substrate in the liquid sample 90 and the enzyme by the first temperature sensor 35.

In one or more embodiments, as described above, the thickness t₁ of the third film 13 is about 16 μm, and the thickness t₂ of the holding film 50 is also about 16 μm. Therefore, the distance D between the flow channel 101 and the sensor chip 30 at the enlarged part 104 is about 32 μm (D=t₁+t₂=32 μm). From the viewpoint of securing highly accurate detection of the reaction heat, the distance D may be 40 μm or less (D≤40 μm).

Further, as described above, in one or more embodiments, the notches 316 are formed in the substrate 31 of the sensor chip 30, and it is possible to connect the connection wirings 55 to the electrodes 34A to 34F via the notches 316 from the lower surface 31b side of the substrate 31. Therefore, there are no inclusions such as solder or conductive paste between the upper surface of the sensor chip 30 and the holding film 50, and it is possible to bring the upper surface of the sensor chip 30 into close contact with the holding film 50 without a step.

Further, since the holding sheets 15A and 15B are arranged side by side at intervals along the width direction of the flow channel 101 as described above, the first and second temperature sensors 35 and 36 are also arranged side by side at intervals along the width direction of the flow channel 101. More specifically, when cutting along the width direction of the flow channel 101, the measuring junction 352 and 362 of the first and second temperature sensors 35 and 36 are arranged side by side at predetermined intervals along the width direction of the flow channel 101 to correspond to the same opening of the channel 101. Therefore, the first and second temperature sensors 35 and 36 can detect the temperatures of the holding sheets 15A and 15B arranged at the same position of the flow channel 101 in the flow direction of the liquid sample 90.

The channel member 10 and the sensor member 20 are not joined by an adhesive or the like, and are separatably fixed by the fixing device 60 in a state of being overlapped with each other. As shown in FIG. 1, FIG. 2 and FIG. 4, the fixing device 60 includes a pair of metal plates 61A and 61B, a rubber heater 62, and a clamp 63. In one or more embodiments, although the fixing device 60 fixes the channel member 10 and the sensor member 20 by utilizing a screw fastening mechanism, the fixing device 60 may fix the channel member 10 and the sensor member 20 by utilizing other mechanisms such as clips.

The pair of metal plates 61A and 61B are plate-shaped members (or heating plates) made of, for example, a metal material such as aluminum. The metal plates 61A and 61B sandwich the channel member 10 and the sensor member 20 which are overlapped with each other. The rubber heater 62 is a heater including a folded rubber sheet capable of covering the metal plates 71A and 71B, and a heat generating resistor embedded in the rubber sheet. The clamp 63 sandwiches and fixes the rubber heater 62 covering the metal plates 61A and 61B sandwiching the channel member 10 and the sensor member 20 from above and below. Instead of the folded rubber heater 72, a pair of plate-shaped heaters separated from each other may be used.

By turning on the rubber heater 62 in a state in which the fixing 60 fixes the channel member 10 and the sensor member 20, it is possible to heat the channel member 10 via the metal plates 61A and 61B. Openings 611 and 621 are respectively formed in the upper metal plate 61A and the rubber heater 62, and the tubular body 18 of the channel member 10 is exposed to the outside of the rubber heat 62 through the openings 611 and 621. Further, both ends of the channel member 10 project from the metal plate 61A and the rubber heater 62, thereby the outlet 103 of the channel member 10 is exposed to the outside.

The calculating device 70 is composed of, for example, an electronic circuit and is electrically connected to the sensor chip 30 via terminals 43A to 43F of the wiring board 40 of the sensor member 20. As shown in FIG. 1, the calculating device 70 includes a calculating unit 71, a storage unit 72, and a display unit 73.

Here, it is known that the reaction heat (temperature change) ΔT generated by the contact reaction between the substrate contained in the liquid sample and the enzyme corresponding to the substrate is proportional to the amount of the substrate. By using this, the calculating unit 71 calculates the amount of the substrate on the basis of the output voltage of the first and second temperature sensors 35 and 36.

Specifically, a table showing the previously measured correspondence relationship between the reaction heat ΔT and the amount of the substrate is stored in advance in the storage unit 72. Then, the calculation unit 71 acquires the output voltages of the first and second temperature sensors 35 respectively, calculates the difference between these output voltages, and then calculates the amount of the substrate corresponding to the difference by referring to the table. Then, the display unit 73 displays the calculation result of the calculation unit 71.

Here, the output voltage of the first temperature sensor 35 indicates the temperature of one holding sheet 15A, and the output voltage of the second temperature sensor 36 indicates the temperature of the other holding sheet 15B. Since these holding sheets 15A and 15B are disposed substantially the same in the flow channel 101, the temperatures of the holding sheets 15A and 15B are almost the same before the contact reaction between the substrate and the enzyme occurs. Therefore, the difference between the output voltage of the first temperature sensor 35 and the output voltage of the second temperature sensor 36 is equivalent to the reaction heat ΔT generated by the contact reaction between the substrate and the enzyme.

In one or more embodiments, as described above, the second electrodes 34B are commonly used as the cold junctions of the first and second thermal sensors 35 and 36. Therefore, only by measuring the potential difference between the first electrode 34A and the third electrode 34C of the sensor chip 30, it is possible to measure the reaction heat ΔT generated by the contact reaction between the substrate and the enzyme.

The calculation unit 71 may calculate the concentration of the substrate in the liquid sample on the basis of the amount of the substrate calculated from the reaction heat ΔT. Alternatively, when a plurality of kinds of enzymes are fixed in the flow channel 101 (see FIG. 9), the calculation unit 71 may calculate the ratio of the plurality of kinds of substrates corresponding to the enzymes. Alternatively, the biosensor 1 may not include the holding sheet 15B and the second temperature sensor 36, and the reaction heat ΔT may be measured on the basis of the time-series data of the output voltage of the first temperature sensor 35.

As described above, in one or more embodiments, since the channel member 10 and the sensor member 20 can be separated from each other, only the channel member 10 is discarded once the biosensor 1 is used, and the sensor member 20, the fixing device 60 and the calculating device 70 can be reused, thereby reducing the cost of the disposable biosensor 1.

In particular, in one or more embodiments, since the sensor member 20 is manufactured by a semiconductor manufacturing process whereas the channel member 10 is manufactured by a low-cost method other than the semiconductor manufacturing process (in one or more embodiments, a method of adhering films), it is possible to further reduce the cost of the disposable biosensor 1.

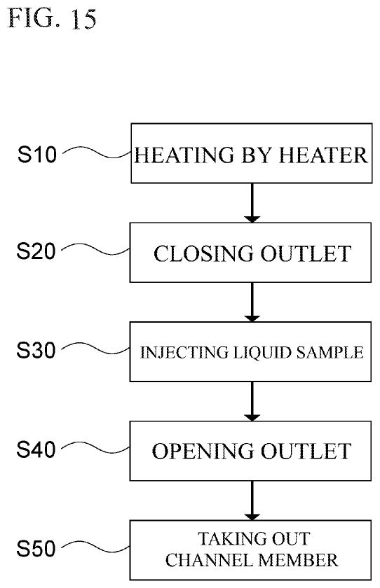

Hereinafter, a method of using the biosensor 1 in one or more embodiments will be described with reference to FIG. 15 and FIG. 16A to FIG. 16C.

FIG. 15 is a process diagram showing a method of using the biosensor in one or more embodiments, FIG. 16A is a cross-sectional view of the step S20 of FIG. 15, and FIG. 16B is a cross-sectional view of the step S30 of FIG. 15, and FIG. 16C is a cross-sectional view of step S40 of FIG. 15.

First, in step S10 of FIG. 15, the rubber heater 62 of the fixing device 60 is turned on to heat the channel member 10. Here, in general, the optimum temperature for the catalytic activity of the enzyme is 35° C. to 40° C. Therefore, although not particularly limited, in one or more embodiments, the channel member 10 is heated by the rubber heater 62 so that the temperature of the channel member 10 is about 38° C. At this time, since the pair of metal plates 61A and 61B sandwich the channel member 10 and the sensor member 20, the rubber heater 62 can uniformly heat the entire channel member 10 via the metal plates 61A and 61B.

Next, in step S20 of FIG. 15, as shown in FIG. 16A, the adhesive tape 80 is attached to the opening 113 (the outlet 103 of the flow channel 101) of the channel member 10 so as to close the opening 113. Instead of the adhesive tape 80, the closing member may be automatically pressed against the opening 113 of the channel member 10.

The before and after relationship between the step S10 and S20 is not particularly limited to the above. The step S10 may be executed after the step S20 is executed, or the step S10 and S20 may be executed simultaneously.

Next, in step S30 of FIG. 15, as shown in FIG. 16B, the liquid sample 90 is injected into the inner hole 181 of the tubular body 18 of the channel member 10. At this time, for example, the liquid sample 90 in the same amount as the total volume in the flow channel 101 is injected into the inner hole 181 of the tubular body 18 by using a syringe. Since the outlet 103 is closed in the above-described step S20, the liquid sample 90 does not flow into the flow channel 101 but still stays in the inner hole 181 of the tubular body 18 in this step S30.

After a predetermined time has elapsed since the liquid sample 90 was filled in the tubular body 18 in the step S30, the adhesive tape 80 is removed from the channel member 10 and the outlet 103 is opened in step S40 of FIG. 15 as shown in FIG. 16C. As a result, the liquid sample 90 held in the inner hole 181 of the tubular body 18 automatically flows into the flow channel 101 without using a pump or the like. Incidentally, in one or more embodiments, before the inflow in this step S40, no other liquid exists in the flow channel 101, only the gas exists.

As described above, in one or more embodiments, since the liquid sample 90 filled in the tubular body 18 is automatically flowed into the flow channel 101 by utilizing the capillary phenomenon without using a pump or the like, it is possible to reduce the cost and size of the biosensor 1.

The predetermined time PT described above is not particularly limited, but is a time sufficient for the temperature of the liquid sample 90 to match the temperature of the channel member 10, and is, for example, 1 seconds to 300 seconds (1 sec PT 300 sec), preferably 1 seconds to 150 seconds (1 sec≤PT≤150 sec), and more preferably 1 seconds to 60 seconds (1 sec≤PT≤60 sec). Instead of the elapse of the predetermined time PT, the step S40 may be executed when the temperature of the liquid sample 90 in the tubular body 18 becomes equal to or higher than the predetermined temperature.

When the liquid sample 90 flows into the flow channel 101 and the liquid sample 90 reaches the enlarged part 104 and the holding sheet 15A and 15B is immersed in the liquid sample 90, the substrate in the liquid sample 90 is brought into contact with the enzyme held in the holding sheet 15A, and the reaction heat (rising heat) ΔT is generated by the contact reaction (contact catalytic reaction) of the substrate and the enzyme. At this time, since the first and second temperature sensors 35 and 36 measure the temperature of the holding sheet 15A and 15B, the calculating device 70 measures the reaction heat ΔT by acquiring the difference between the signals outputted from the first and second temperature sensors 35 and 36 and calculates the quantity of the substrate on the basis of the reaction heat ΔT.

In one or more embodiments, since the enzyme is held in the holding sheet 15A, the reaction heat generated by the contact reaction between the substrate in the liquid sample 90 and the enzyme is stored in the holding sheet 15A and then diffused slowly. Therefore, it is possible to secure a state long in which the reaction heat can be detected by the first temperature sensor 35 and it is possible to detect the reaction heat with high accuracy. Therefore, even when the channel member 10 and the sensor member 20 are separated and the third film 13 and the holding film 50 are interposed between the flow channel 101 and the sensor chip 30, it is possible to detect the reaction heat ΔT by the first temperature sensor 35.

Further, in one or more embodiments, since the holding sheet 15A is made of a nonwoven fabric, the holding sheet 15A has a large number of communication paths that penetrate from the upper surface 151 to the lower surface 152, and the enzyme is held in this holding sheet 15A. Therefore, since the contact area between the liquid sample 90 and the enzyme increases, it is possible to increase the reaction heat ΔT due to the contact reaction between the substrate in the liquid sample 90 and the enzyme, and it is possible to secure the state longer in which the reaction heat ΔT can be detected by the first temperature sensor 35.

Further, since the holding sheet 15A has a large number of communication paths that penetrate from the upper surface 151 to the lower surface 152, the holding sheet 15A does not hinder the heat transfer of the reaction heat ΔT to the first temperature sensor 35.

In one or more embodiments, since the flow channel 101 and the sensor chip 30 are in contact with each other in the plane of the third film 13 and the holding film 50, the thermal resistance between them is small. Therefore, the first thermal sensor 35 can accurately detect the minute reaction heat generated in the holding sheet 15A.

Incidentally, since the thickness t₁ of the third film 13 is extremely thinner than the other members 11, 12 and 14 constituting the channel member 10, the third film 13 swells with the inflow of the liquid sample 90 into the flow channel 101. Since, due to this expansion, the third film 13 and the holding film 50 are brought into close contact with each other, the thermal resistance between the flow channel 101 and the sensor chip 30 is further reduced.

Further, in one or more embodiments, the notches 316 are formed in the substrate 31 of the sensor chip 30, and the connecting wires 55 are connected from the surface 31b side of the substrate 31 to the electrodes 34A-34F of the sensor chip 30 via the notches 316. Therefore, there are no inclusions such as solder or conductive pastes between the upper surface of the sensor chip 30 and the holding film 50, and the upper surface of the sensor chip 30 can be brought into close contact with the holding film 50 without a step.

Further, as described above, the holding sheets 15A and 15B are arranged side by side at predetermined intervals along the width direction of the flow channel 101, and the first and second temperature sensors 35 and 36 are arranged to respectively correspond to the holding sheets 15A and 15B. Therefore, since the first and second temperature sensors 35 and 36 can detect the temperatures of the holding sheets 15A and 15B under substantially the same conditions, the reaction heat ΔT corresponding to the difference between these can be accurately measured.

For example, when the first and second temperature sensors 35 and 36 detect the same temperature without disposing the holding sheet 15B before the fluid sample 90 flowing into the flow channel 101, the holding sheet 15B may be omitted.

After the above inspection of the liquid sample 90 is completed, in step S50 of FIG. 15, the biosensor 10 is disassembled to take out the channel member 10 from the biosensor 1. Specifically, the clamp 73 of the fixing device 70 is loosened, and the channel member 10 is taken out from between the plates 61A and 61B. At this time, in one or more embodiments, since the channel member 10 and the sensor member 20 are only overlapped with each other and are not joined by an adhesive or the like, it is possible to easily separate the channel member 10 and the sensor member 20.

Then, the channel member 10 removed from the biosensor 1 is discarded. Therefore, in the biosensor 1 of one or more embodiments, work such as cleaning of the flow channel 101 becomes unnecessary, and the biosensor 1 is also excellent in hygiene. On the other hand, since the members other than the channel member 10 (i.e., the sensor member 20, the fixing device 60, and the calculating device 70) are reused, it is possible to reduce the cost of the disposable biosensor 1.

Embodiments heretofore explained are described to facilitate understanding of the present disclosure and are not described to limit the disclosure. It is therefore intended that the elements disclosed in the above embodiments include all design changes and equivalents to fall within the technical scope of the disclosure.

For example, a plurality of kinds of enzymes may be fixed in the same holding sheet. As a specific example in this case, when the substrate is glucose and the enzyme is glucose oxidase, catalase may be held in the holding sheet in addition to this glucose oxidase. Since hydrogen peroxide is generated by the contact reaction of glucose and glucose oxidase, it is possible to increase the reaction heat ΔT by a further contact reaction of this hydrogen peroxide with catalase.

Further, a liquid sample may contain an enzyme, and a substrate corresponding to the enzyme may be fixed to the holding sheet. As a specific example in this case, the enzyme is acidic phosphatase and the substrate is 1-naphthyl phosphate.

Further, the liquid sample may be a liquid other than a body fluid, and may be, for example, a liquid obtained from vegetables, fruits, or seaweed or the like.

Further, although, in the embodiments described above, the biosensor 1 in which the channel member 10 and the sensor member 20 can be separated from each other has been described, the configuration of the biosensor is not particularly limited thereto. The holding sheet 15 may be applied to the biosensor in which the channel member and the sensor member are integrated, and it is also possible to detect the reaction heat with high accuracy in this case.

Although the disclosure has been described with respect to only a limited number of embodiments, those skilled in the art, having benefit of this disclosure, will appreciate that various other embodiments may be devised without departing from the scope of the present disclosure. Accordingly, the scope of the disclosure should be limited only by the attached claims.

### REFERENCES SIGNS LIST

1 BIOSENSOR
10 CHANNEL MEMBER
101 FLOW CHANNEL
11 FIRST FILM
111 HYDROPHILIZED SURFACE
12 SECOND FILM
121 HYDROPHILIZED SURFACE
13 THIRD FILM
131 ADHESIVE LAYER
14 SPACER
15 HOLDING SHEET
16 RESIN MATERIAL
18 TUBULAR BODY
20 SENSOR MEMBER
30 SENSOR CHIP
31 SUBSTRATE
313 SECOND Si LAYER
316 NOTCH
33A and 33B BEAM PART
34A to 34F ELECTRODE
35 FIRST TEMPERATURE SENSOR
352 MEASURING JUNCTION
36 SECOND TEMPERATURE SENSOR
40 WIRING BOARD
44 OPENING
50 HOLDING FILM
51 ADHESIVE LAYER
55 CONNECTION WIRING
60 FIXING DEVICE
61A and 61B METAL PLATE
62 RUBBER HEATER
70 CALCULATING DEVICE
80 ADHESIVE TAPE
90 LIQUID SAMPLE

1. A biosensor comprising:
a flow channel through which a liquid sample flows, the liquid sample containing a specific component;
a holding sheet that is disposed in the flow channel and holds a substance corresponding to the specific component; and
a first temperature sensor that is disposed to correspond to the holding sheet and detects a reaction heat generated by a contact reaction between the specific component and the corresponding substance, wherein
the biosensor acquires information on the specific component based on the reaction heat.

2. The biosensor according to claim 1, wherein
the holding sheet has two surfaces and includes communication paths that pass from one of the two surfaces to the other of the two surfaces.

3. The biosensor according to claim 1, wherein
the holding sheet includes a cloth, paper, a porous body, or a net-like body.

4. The biosensor according to claim 1, wherein
the holding sheet holds a resin material that contains the corresponding substance and gets cured while entering the holding sheet.

5. The biosensor according to claim 1, further comprising:
a channel sheet that comprises the flow channel; and
a sensor plate that comprises the first temperature sensor, wherein
the channel sheet is separatable from the sensor plate.

6. The biosensor according to claim 5, wherein
the channel sheet further comprises a channel wall that holds the holding sheet and defines the flow channel,
the sensor plate comprises:
a sensor chip that includes the first temperature sensor; and
a holding film on which the sensor chip is mounted,
the channel wall and the holding film are disposed between the holding sheet and the first temperature sensor, and
the channel wall is not bonded to the holding film, and the channel sheet is separatable from the sensor plate.

7. The biosensor according to claim 5, further comprising:
a fixing device that fixes the channel sheet and the sensor plate, wherein
the fixing device comprises:
a pair of heating plates that sandwich the channel sheet and the sensor plate stacked on each other.

8. The biosensor according to claim 1, wherein
the flow channel includes an enlarged part whose inner diameter is enlarged toward the first temperature sensor as compared with other part of the flow channel, and
the holding sheet is disposed in the enlarged part.

9. The biosensor according to claim 8, further comprising:
a channel wall that holds the holding sheet and defines the flow channel, wherein
a part of an inner surface of the channel wall is a hydrophilized surface that is subjected to a hydrophilic treatment, and
a part of an inner surface of the channel wall in the enlarged part has hydrophobicity as compared with the hydrophilized surface.

10. The biosensor according to claim 1, further comprising:
a channel sheet that comprises the flow channel, wherein
the channel sheet comprises:
a first film that has a hydrophilized surface;
a second film that has a hydrophilized surface and has a first opening in a part of a region corresponding to the flow channel;
a spacer that has a groove having a shape corresponding to the flow channel and is attached to the first and second films to be disposed between the first and second films; and
a third film that has an adhesive layer on a main surface of the third film and is attached to the second film to close the first opening, and
the holding sheet is attached to the adhesive layer of the third film.

11. The biosensor according to claim 1, further comprising:
a sensor chip that comprises:
a beam part in which the first temperature sensor is formed;
a board that includes a semiconductor layer forming a part of the first temperature sensor; and
an electrode that is disposed on one side of the board and is electrically connected to the first temperature sensor, wherein
a part of the board corresponding to the electrode is removed such that the electrode is exposed on other side of the board.

12. The biosensor according claim 1, further comprising:
a sensor plate that comprises:
the first temperature sensor;
a sensor chip that includes the first temperature sensor;
a holding film that holds the sensor chip;
a wiring board that has a second opening that accommodates the sensor chip, the holding film being attached to the wiring board to close the second opening; and
a connecting body that connects an electrode of the sensor chip and the wiring of the wiring board.

13. The biosensor according to claim 1, further comprising:
a tubular body disposed at an inlet of the flow channel and communicating with the flow channel, wherein
an inner hole of the tubular body has a volume equal to or larger than a volume of the flow channel.

14. The biosensor according to claim 1, wherein
the specific component includes one of a substrate and an enzyme, and
the corresponding substance includes another of the enzyme and the substrate.

15. The biosensor according to claim 1, further comprising:
a second temperature sensor that is disposed to correspond to a part of the flow channel in which the corresponding substance is not disposed, wherein
the first temperature sensor and the second temperature sensor are disposed along a width direction of the flow channel.

16. The biosensor according to claim 1, further comprising:
a second temperature sensor that is disposed to correspond to a part of the flow channel in which the corresponding substance is not disposed; and
the calculating device that calculates an amount of the specific component based on a first detection result by the first temperature sensor and a second detection result by the second temperature sensor.

17. A channel sheet that is used in a biosensor, comprising:
a flow channel through which a liquid sample flows, the liquid sample containing a specific component; and
a holding sheet that is disposed in the flow channel and holds a substance corresponding to the specific component, wherein
the biosensor acquires information on the specific component based on a reaction heat generated by a contact reaction between the specific component and the corresponding substance.

18. A method for using the biosensor according to claim 13, comprising:
(a) closing an outlet of the flow channel;
(b) injecting the liquid sample into the inner hole of the tubular body;
(c) opening the outlet; and
(d) detecting the reaction heat by the first temperature sensor.

19. The method according to claim 18, wherein
the step (c) is performed after the step (b) is finished.

20. The method according to claim 18, further comprising:
(e) removing the channel sheet from the biosensor.
